(19) United States
(12) Patent Application Publication
(10) Pub. No.: US 2022/0026391 A1
(43) Pub. Date: Jan. 27, 2022

(54) ESTIMATING COIL IMPLANT DEPTH FOR WIRELESS POWER TRANSFER

(71) Applicant: Medtronic, Inc., Minneapolis, MN (US)

(72) Inventors: Eric A. SCHILLING, Ham Lake, MN (US); Erin N. REISFELD, Minneapolis, MN (US); David J. PEICHEL, Minneapolis, MN (US)

(21) Appl. No.: 16/937,685

(22) Filed: Jul. 24, 2020

(51) Int. Cl. G01N 27/72; A61B 5/00; A61B 5/145

(52) U.S. Cl. CPC G01N 27/72; A61B 5/14503; A61B 5/0031

(57) ABSTRACT

A method of estimating a depth of an implanted receiving coil of a transcutaneous energy transfer system (TETS) includes aligning the implanted receiving coil with an external transmission coil. A coupling coefficient between the implanted receiving coil and the external transmission coil is estimated based on a first transfer function and operating conditions. The depth of the implanted receiving coil is estimated from the estimated coupling coefficient based on a second transfer function.

### CROSS-REFERENCE TO RELATED APPLICATION

n/a

### FIELD

The present technology is generally related to transcutaneous energy transfer systems (TETS) and in particular, a method of estimating a depth of an implanted receiving coil.

### BACKGROUND

In transcutaneous energy transfer systems (TETS) the efficiency of transfer between an implanted receiving coil and an external transmission is determined by the coupling coefficient between the two coils. However, the coupling coefficient may change as the patient moves, or if the depth of the implanted receiving coil changes as a result of the patient gaining or losing weight.

### SUMMARY

The techniques of this disclosure generally relate to transcutaneous energy transfer systems (TETS) and in particular, a method of estimating a depth of an implanted receiving coil.

In one aspect, a method of estimating a depth of an implanted receiving coil of a transcutaneous energy transfer system (TETS) includes aligning the implanted receiving coil with an external transmission coil. A coupling coefficient between the implanted receiving coil and the external transmission coil is estimated based on a first transfer function and operating conditions. The depth of the implanted receiving coil is estimated from the estimated coupling coefficient based on a second transfer function.

In another aspect of this embodiment, the method further includes estimating the coupling coefficient periodically over a predetermined period of time following a time of implantation of the implanted receiving coil.

In another aspect of this embodiment, the method further includes recording and compiling of the estimated coupling coefficient over the predetermined period of time.

In another aspect of this embodiment, recording and compiling includes creating a histogram of the estimated coupling coefficient, and wherein if based on the histogram at a time of re-estimation, a maximum estimated coupling coefficient deviates by a predetermined threshold from a historical maximum coupling coefficient of the histogram, the depth of the implanted receiving coil is re-estimated.

In another aspect of this embodiment, if based on the histogram at a time of re-estimation, a predetermined percentage of a distribution of the created histogram is less than or greater than a historical maximum coupling coefficient, the depth of the implanted receiving coil is re-estimated.

In another aspect of this embodiment, the predetermined percentage is between 95-99%.

In another aspect of this embodiment, the method further includes displaying on a display in communication with the implanted receiving coil the created histogram and the estimated depth of the implanted receiving coil.

In another aspect of this embodiment, the method further includes calibrating a coil alignment indicator on an external controller in communication with the implanted receiving coil after estimating the depth of the implanted receiving coil.

In another aspect of this embodiment, aligning the implanted receiving coil with the external transmission coil occurs at a time of implantation.

In another aspect of this embodiment, estimating the coupling coefficient between the implanted receiving coil and the external transmission coil based on the first transfer function occurs at the time of implantation.

In one aspect, a method of estimating a depth of an implanted receiving coil of a transcutaneous energy transfer system (TETS) includes aligning the implanted receiving coil with an external transmission coil. A coupling coefficient between the implanted receiving coil and the external transmission coil is estimated based on a first transfer function. A depth of the implanted receiving coil is measured with one from the group consisting of a computerized tomography scanner and a physical measurement. The coupling coefficient is periodically estimated following a time of implantation over a predetermined period of time. A histogram of the estimated coupling coefficient is created over the predetermined period of time. The depth of the implanted receiving coil is re-estimated if based on the histogram, one from the group consisting of: the estimated coupling coefficient at a time of re-estimation deviates by a predetermined threshold from a historical maximum coupling coefficient of the created histogram and a predetermined percentage of a distribution of the created histogram is less than or greater than a historical maximum coupling coefficient.

In another aspect of this embodiment, the predetermined percentage is between 95-99%.

In another aspect of this embodiment, the method includes displaying on a display in communication with the implanted receiving coil the created histogram and the measured depth of the implanted receiving coil.

In another aspect of this embodiment, the method includes calibrating a coil alignment indicator after measuring the depth of the implanted receiving coil.

In another aspect of this embodiment, aligning the implanted receiving coil with the external transmission coil occurs at the time of implantation.

In another aspect of this embodiment, measuring the depth of the implanted receiving coil occurs at the time of implantation.

In one aspect, a controller in communication with an implantable blood pump includes processing circuitry configured to: estimate a coupling coefficient between an implanted receiving coil and an external transmission coil based on a first transfer function at a time of implantation; estimate a depth of the implanted receiving coil from the estimated coupling coefficient based on a second transfer function at the time of implantation; periodically estimate the coupling coefficient following the time of implantation over a predetermined period of time; create a histogram of the estimated coupling coefficient over the predetermined period of time; re-estimate the depth of the implanted receiving coil if based on this histogram, one from the group consisting of: the estimated coupling coefficient at a time of re-estimation deviates by a predetermined threshold from a historical maximum coupling coefficient of the created histogram; a predetermined percentage of a distribution of the created histogram is less than or greater than a historical maximum coupling coefficient and display on a display in communication with the implanted receiving coil the created histogram and the estimated depth of the implanted receiving coil.

In another aspect of this embodiment, the predetermined percentage is between 95-99%.

In another aspect of this embodiment, estimating the coupling coefficient between the implanted receiving coil and the external transmission coil based on the first transfer function at the time of implantation occurs after the implanted receiving coil and the external transmission coil are aligned at the time of implantation.

In another aspect of this embodiment, the predetermined threshold is between 10-30 percent.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is an internal system view of an implantable blood pump with a TETS receiver source constructed in accordance with the principles of the present application;

FIG. 2 is an external view of a TETS transmitter and a controller of the system shown in FIG. 1; and

FIG. 3 is a flow chart showing the steps for estimating a depth of an implanted receiving coil; and

FIG. 4 is a histogram showing historical coupling coefficients in accordance with the method shown in FIG. 3.

### DETAILED DESCRIPTION

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

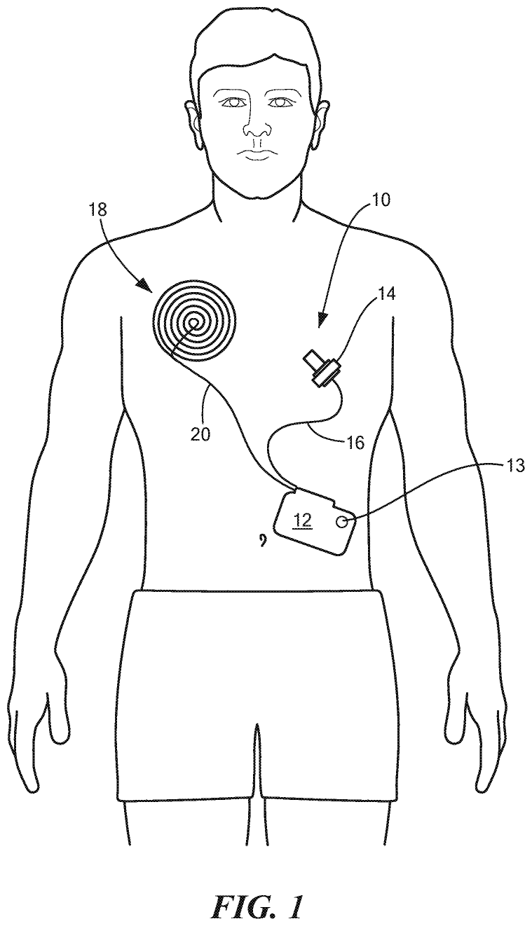

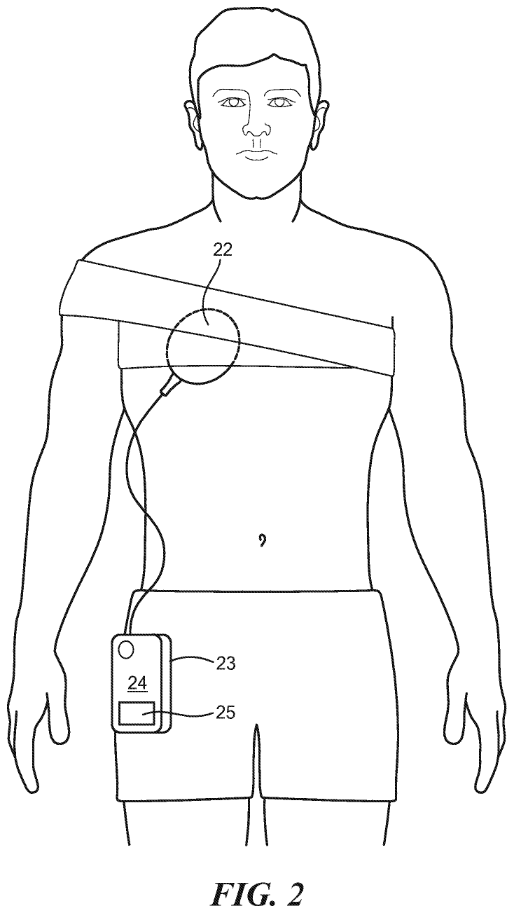

Referring now to the drawings in which like reference designators refer to like elements there is shown in FIGS. 1 and 2 an exemplary mechanical circulatory support device (“MCSD”) constructed in accordance with the principles of the present application and designated generally as “10.” The MCSD 10 may be fully implantable within a patient, whether human or animal, which is to say there are no percutaneous connections between the implanted components of the MCSD 10 and the components outside of the body of the patient. In the configuration shown in FIG. 1, the MCSD 10 includes an internal controller 12 implanted within the body of the patient. The internal controller 12 includes a control circuit having processing circuitry configured to control operation of an implantable blood pump 14. The internal controller 12 may include an internal power source 13, configured to power the components of the controller and provide power to one or more implantable medical devices, for example, the implantable blood pump, such as a ventricular assist device (“VAD”) 14 implanted within the left ventricle of the patient's heart. The power source 13 may include a variety of different types of power sources including an implantable battery. VADs 14 may include centrifugal pumps, axial pumps, or other kinds electromagnetic pumps configured to pump blood from the heart to blood vessels to circulate around the body. One such centrifugal pump is the HVAD and is shown and described in U.S. Pat. No. 7,997,854, the entirety of which is incorporated by reference. One such axial pump is the MVAD and is shown and described in U.S. Pat. No 8,419,609, the entirety of which is incorporated herein by reference. In an exemplary configuration, the VAD 14 is electrically coupled to the internal controller 12 by one or more implanted conductors 16 configured to provide power to the VAD 14, relay one or more measured feedback signals from the VAD 14, and/or provide operating instructions to the VAD 14.

Continuing to refer to FIG. 1, a receiving or internal coil 18 may also be coupled to the internal controller 12 by, for example, one or more implanted conductors 20. In an exemplary configuration, the receiving coil 18 may be implanted subcutaneously proximate the thoracic cavity, although any subcutaneous position may be utilized for implanting the receiving coil 18. The receiving coil 18 is configured to be inductively powered through the patient's skin by a transmission or external coil 22 (seen in FIG. 2) disposed opposite the receiving coil 18 on the outside/exterior of the patient's body. For example, as shown in FIG. 2, a transmission coil 22 may be coupled to an external controller 23 having a power source 24, for example, a portable battery carried by the patient or wall power, and a coil alignment indicator 25. In one configuration, the battery is configured to generate a radiofrequency signal for transmission of energy from the transmission coil 22 to the receiving coil 18. The receiving coil 18 may be configured for transcutaneous inductive communication with the transmission coil 22 to define a transcutaneous energy transfer system (TETS) that receives power from the transmission coil 22.

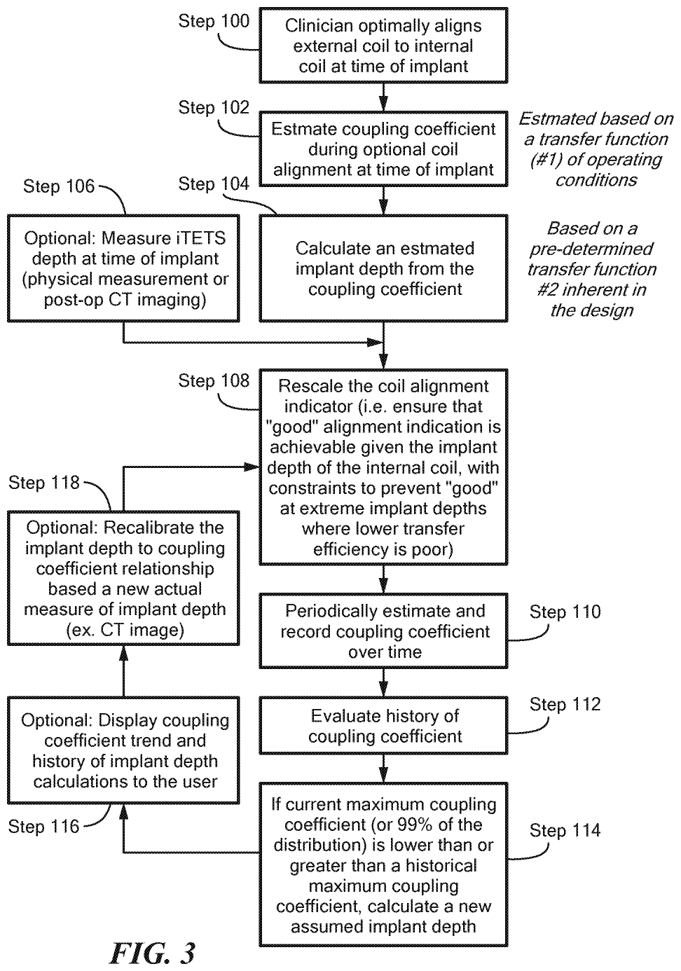

Referring now to FIG. 3, in which a method of estimating a depth of the implanted receiving coil 18 is shown. In one configuration, at the time of implantation of the receiving coil, the clinician aligns the transmission coil 22 with the implanted receiving coil 18 for transmission of TETS power (Step 100), which can be achieved via fluoroscopy or via a palpation approach. A coupling coefficient between the receiving coil 18 and the transmission coil 22 is then calculated based on a first transfer function and operating conditions between the receiving coil 18 and the transmission coil 22 (Step 102). In particular, the first transfer function calculation uses the frequency, the current in the transmission coil 22, output and input power, and the efficiency to estimate the coupling coefficient. Once the coupling coefficient is estimated, the depth of the implanted receiving coil 18 may be estimated with a second transfer function different than the first transfer function (Step 104). For example, the second transfer function estimates the subcutaneous depth of receiving coil 18 based on, for example, the radial offset between the two coils 18 and 22 and the coupling coefficient, with no radial offset for the depth estimation. In one configuration, both the first and second transfer functions are based on electromagnetic and circuit simulations but may be derived empirically by bench testing. Optionally, the depth of the implanted coil 18 can be measured by, for example, an imaging technique such as computerized tomography scan at the time of implantation or other physical measurements (Step 106).

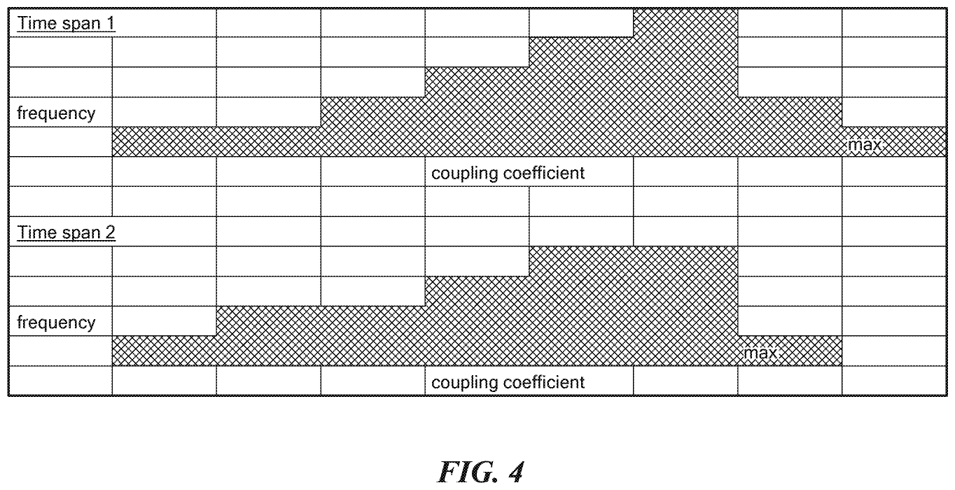

Continuing to refer to FIG. 3, the coil alignment indicator 25, which may be present on the external controller 23 and in communication with the internal controller 12 and the implanted receiving coil 18, may be calibrated based on the operating conditions between the transmission coil 22 and the implanted receiving coil 18 (Step 108). For example, the coil alignment indicator 25 may be set to “good” following the alignment of the coils and verification of operating conditions. Following the implantation of the implanted receiving coil, the coupling coefficient may be periodically estimated over a predetermined period of time (Step 110). For example, the coupling coefficient may be re-estimated periodically over a month or a few months and the estimated coupling coefficients are compiled and recorded over the predetermined period of time. That is, the coupling coefficients over the predetermined period of time may be re-estimated, compiled, and presented in a histogram and evaluated, as shown in FIG. 4 (Step 112). If based on the histogram at a time of re-estimation, a maximum estimated coupling coefficient deviates by a predetermined threshold from a historical maximum coupling coefficient of the histogram, the depth of the implanted receiving coil is re-estimated. (Step 114). In particular, when the coupling coefficient is re-estimated, for example a month after implantation, if the estimated maximum coupling coefficient is lower or higher by a predetermined threshold, for example, 10-30%, compared to the historical maximum evaluated from the histogram (or the value stored from the time of implant), then the depth of the implanted coil is re-estimated based on the re-estimated coupling coefficient. One possible explanation for the historical coupling coefficient being lower than the coupling coefficient at the time of implantation is that the patient may have gained weight which may increase the amount of adipose tissue between the implanted receiving coil 18 and the transmission coil. Conversely, if the patient loses weight, the coupling coefficient may also change as the amount of adipose tissue may decrease. Moreover, if based on the histogram at a time of re-estimation, a predetermined percentage of a distribution of the created histogram, for example, 95-99%, is less that historical maximum coupling coefficient, the depth of the implanted receiving coil is re-estimated. In particular, if between 95-99% of the historical distribution during the predetermined time period is less than the historical maximum coupling coefficient at the time of estimation, then the depth of the implanted is re-estimated as the historical data from the histogram indicates that the coupling coefficient has changed since implantation. Optionally, the controller 23 may display the created histogram and the estimated depth of the implanted receiving coil (Step 116). For example, a clinician may be able to recall the histogram from memory on the external controller 23, and display the histogram to see the coupling coefficient trends over time. Optionally, if it is determined that a new implanted receiving coil 18 depth is to be calculated, and if the implant depth is calculated using a CT scan, then the coil alignment indicator 25 may be recalibrated based on the actual implant depth measured by the CT Scan (Step 118).

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A method of estimating a depth of an implanted receiving coil of a transcutaneous energy transfer system (TETS), comprising:
aligning the implanted receiving coil with an external transmission coil;
estimating a coupling coefficient between the implanted receiving coil and the external transmission coil based on a first transfer function and operating conditions; and
estimating the depth of the implanted receiving coil from the estimated coupling coefficient based on a second transfer function.

2. The method of claim 1, further including estimating the coupling coefficient periodically over a predetermined period of time following a time of implantation of the implanted receiving coil.

3. The method of claim 2, further including recording and compiling of the estimated coupling coefficient over the predetermined period of time.

4. The method of claim 3, wherein recording and compiling includes creating a histogram of the estimated coupling coefficient, and wherein if based on the histogram at a time of re-estimation, a maximum estimated coupling coefficient deviates by a predetermined threshold from a historical maximum coupling coefficient of the histogram, the depth of the implanted receiving coil is re-estimated.

5. The method of claim 3, wherein if based on the histogram at a time of re-estimation, a predetermined percentage of a distribution of the created histogram is less than or greater than a historical maximum coupling coefficient, the depth of the implanted receiving coil is re-estimated.

6. The method of claim 5, wherein the predetermined percentage is between 90-99.9%.

7. The method of claim 6, further including displaying on a display in communication with the implanted receiving coil the created histogram and the estimated depth of the implanted receiving coil.

8. The method of claim 1, further including calibrating a coil alignment indicator on an external controller in communication with the implanted receiving coil after estimating the depth of the implanted receiving coil.

9. The method of claim 1, wherein aligning the implanted receiving coil with the external transmission coil occurs at a time of implantation.

10. The method of claim 9, wherein estimating the coupling coefficient between the implanted receiving coil and the external transmission coil based on the first transfer function occurs at the time of implantation.

11. A method of estimating a depth of an implanted receiving coil of a transcutaneous energy transfer system (TETS), comprising:
aligning the implanted receiving coil with an external transmission coil;
estimating a coupling coefficient between the implanted receiving coil and the external transmission coil based on a first transfer function;
measuring the depth of the implanted receiving coil with one from the group consisting of a computerized tomography scanner and a physical measurement;
periodically estimating the coupling coefficient following a time of implantation over a predetermined period of time;
creating a histogram of the estimated coupling coefficient over the predetermined period of time; and
the depth of the implanted receiving coil is re-estimated if based on the histogram, one from the group consisting of:
the estimated coupling coefficient at a time of re-estimation deviates by a predetermined threshold from a historical maximum coupling coefficient of the created histogram; and
a predetermined percentage of a distribution of the created histogram is less than or greater than a historical maximum coupling coefficient.

12. The method of claim 11, wherein the predetermined percentage is between 90-99.9%.

13. The method of claim 11, further including displaying on a display in communication with the implanted receiving coil the created histogram and the measured depth of the implanted receiving coil.

14. The method of claim 11, further including calibrating a coil alignment indicator after measuring the depth of the implanted receiving coil.

15. The method of claim 11, wherein aligning the implanted receiving coil with the external transmission coil occurs at the time of implantation.

16. The method of claim 11, wherein measuring the depth of the implanted receiving coil occurs at the time of implantation.

17. A controller in communication with an implantable blood pump, comprising:
processing circuitry configured to:
estimate a coupling coefficient between an implanted receiving coil and an external transmission coil based on a first transfer function at a time of implantation;
estimate a depth of the implanted receiving coil from the estimated coupling coefficient based on a second transfer function at the time of implantation;
periodically estimate the coupling coefficient following the time of implantation over a predetermined period of time;
create a histogram of the estimated coupling coefficient over the predetermined period of time;
re-estimate the depth of the implanted receiving coil if based on this histogram, one from the group consisting of:
the estimated coupling coefficient at a time of re-estimation deviates by a predetermined threshold from a historical maximum coupling coefficient of the created histogram;
a predetermined percentage of a distribution of the created histogram is less than or greater than a historical maximum coupling coefficient; and
display on a display in communication with the implanted receiving coil the created histogram and the estimated depth of the implanted receiving coil.

18. The controller of claim 17, wherein the predetermined percentage is between 95-99%.

19. The controller of claim 17, wherein estimating the coupling coefficient between the implanted receiving coil and the external transmission coil based on the first transfer function at the time of implantation occurs after the implanted receiving coil and the external transmission coil are aligned at the time of implantation.

20. The controller of claim 17, wherein the predetermined threshold is between 10-30 percent.
